SEQUENCE LISTING (18 sequences)

SEQ ID NO: 1 - DNA, 2301 bp, Artificial Sequence
  Description of Artificial Sequence Synthetic polynucleotide
atggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctccggtaccgcggaggatcccgccgagcccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattcgaatttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 2 - protein, 766 aa, Artificial Sequence
  Description of Artificial Sequence Synthetic polypeptide
MEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSGTAEDPAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFEFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - DNA, 57 bp, Artificial Sequence
  Description of Artificial Sequence Synthetic oligonucleotide
atggagtttgggctgagctggctttttcttgtggctattttaaaaggtgtccagtgc

SEQ ID NO: 4 - protein, 19 aa, Artificial Sequence
  Description of Artificial Sequence Synthetic peptide
MEFGLSWLFLVAILKGVQC

SEQ ID NO: 5 - DNA, 747 bp, Artificial Sequence
  Description of Artificial Sequence Synthetic polynucleotide
tctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcaccgtctcc

SEQ ID NO: 6 - protein, 249 aa, Artificial Sequence
  Description of Artificial Sequence Synthetic polypeptide
SRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVS

SEQ ID NO: 7 - DNA, 696 bp, Artificial Sequence
  Description of Artificial Sequence Synthetic polynucleotide
gagcccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 232 aa, Artificial Sequence
  Description of Artificial Sequence Synthetic polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 423 bp, Artificial Sequence
  Description of Artificial Sequence Synthetic polynucleotide
tttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaacctgcatatacattatattcattaattcagccttccaggaagtctggatccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 10 - protein, 141 aa, Artificial Sequence
  Description of Artificial Sequence Synthetic polypeptide
FLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 11 - DNA, 339 bp, Artificial Sequence
  Description of Artificial Sequence Synthetic polynucleotide
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 12 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 19 aa, Artificial Sequence
  Description of Artificial Sequence Synthetic peptide
MEFGLSWLFLVAILKGVQC

SEQ ID NO: 14 - protein, 128 aa, Artificial Sequence
  Description of Artificial Sequence Synthetic polypeptide
SRDILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKRADAAPTVSIFPG

SEQ ID NO: 15 - protein, 8 aa, Artificial Sequence
  Description of Artificial Sequence Synthetic peptide
SGGGGSGG

SEQ ID NO: 16 - protein, 113 aa, Artificial Sequence
  Description of Artificial Sequence Synthetic polypeptide
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 17 - DNA, 21 bp, Artificial Sequence
  Description of Artificial Sequence Synthetic primer
accatggagtttgggctgagc

SEQ ID NO: 18 - DNA, 23 bp, Artificial Sequence
  Description of Artificial Sequence Synthetic primer
gcatgctaacgcgtttagcgagg

(19) United States
(12) Patent Application Publication
(10) Pub. No.: US 2018/0100016 A1
(43) Pub. Date: Apr. 12, 2018

(54) USE OF CAR-MODIFIED HUMAN NATURAL KILLER CELLS TO TREAT CANCER

(71) Applicant: Xiaotong Song, Pearland, TX (US)

(72) Inventor: Xiaotong Song, Pearland, TX (US)

(21) Appl. No.: 15/838,406

(22) Filed: Dec. 12, 2017

Related U.S. Application Data
(60) Provisional application No. 62/437,690, filed on Dec. 22, 2016.

(51) Int. Cl. C07K 16/28; A61K 35/17; A61P 35/00; C07K 14/725; C07K 14/705; C07K 16/30; C07K 14/47

(52) U.S. Cl. CPC C07K 16/2803; A61K 38/00; A61P 35/00; C07K 14/7051; C07K 14/70503; C07K 16/3084; C07K 14/4705; C07K 14/70521; C07K 2317/622; C07K 2317/66; C07K 2317/565; C07K 2317/567; C07K 2319/02; C07K 2319/03; A61K 35/17

(57) ABSTRACT

The invention provides a Natural Killer Cell, which may be a NK92 cell that is modified to express one or more types of Chimeric Antigen Receptor (CAR) on its surface, and administering said cell to a subject for a cancer treatment. Said engineered CAR comprises an antigen binding domain, which may bind CD19 or GD2, a transmembrane domain, a co-stimulatory signaling region, which may be all or a portion or variant of 2B4, and a signaling domain, which may be all or a portion or variant of CD3ζ.

### FIELD OF THE INVENTION

The invention relates to modified Natural Killer cell which expresses one or more particular engineered Chimeric Antigen Receptors (CARs) on its surface.

### BACKGROUND

The disclosure of International application WO2015142675 (Novartis) incorporated by reference. (Introduction to NK cells) NK cells are a class of innate lymphocytes which mediate innate cellular immune responses by recognition and lysis of virus infected cells and tumor cells. NK cells originate in the bone marrow, where they are derived from the common lymphocyte progenitor (other CLP derived cells are T cells, B cells, and some Dendritic Cells), and can comprise up to 10% of the total lymphocyte population in the body. They are defined as being CD3−, CD16+, and CD56+, although variations in CD56 expression exist, and may influence the nature of the NK cell response to target cells. Unlike T and B lymphocytes, which can also recognize viral infected and tumor cells. NK cells do not undergo genetic rearrangement of their receptors to achieve recognition of target cells. Rather, NK cells express a host of activating and inhibitory receptors on their surface that interact with healthy and infected or tumor cells in order to mediate recognition and effector functions. Thus NK cells do not need to undergo selection and clonal expansion before mounting an immune response to infected, damaged, or tumor cells, and do not recognize specific antigens like B or T cells.

NK cells produce intracellular granules containing perforin and granzymes, which upon NK receptor recognition of target cells are exocytosed to mediate apoptosis of the target cell. Other lymphocytes (T cells and B cells) do not produce such granules. In addition, NK cells express cytokines upon activation, including IFNγ, that promote an inflammatory immune response. Outside their role in recognition and effector response against infected or abnormal cells, NK cells also play roles in processes such as humoral immune response regulation and fetal development.

(Introduction to NK cell receptor) NK cells express a variety of markers and receptors on their cell surface, including such as to characterize them as CD3−, CD16+, and CD56+. In addition, numerous activating and inhibitory have also been identified. Said receptors may be activated by their corresponding ligands to trigger cytotoxic effects against target cells, as well as to secrete cytokines in regulating immune responses. Unlike other innate cells, which recognize pathogen associated molecular patterns (PAMPS) or danger associated molecular patterns (DAMPS) expressed by infecting organisms, NK cells survey cells for alterations in expression of normal surface proteins (missing or altered self). A major class of lymphocyte receptors, the killer cell immunoglobulin-like receptors (KIR), are expressed on NK cells, and interact with human leukocyte antigens (HLA) class I molecules expressed on most healthy cells. HLA-I/KIR interaction primarily inhibits the cytolytic killing of the HLA-I expressing cell by NK cells, although some activating KIRs are also expressed by NK cells. Downregulation of HLAI by virally infected or tumor cells leads to a loss of HLA-I/KIR inhibitory signaling in NK cells, contributing to NK activation and lysis of the target cell.

Natural cytotoxicity receptors (NCRs) comprise a second class of receptors expressed on the NK cell surface. Many NCRs, such as NKp46 and NKp44 are activating receptors. These receptors are capable of recognizing both virally expressed proteins at the infected cell surface, as well as cellular proteins that are expressed at the cell surface only in cases of transformation to tumor cells, intracellular damage, or cell stress. Not all of these changes to surface protein expression result in NCR activation, as like KIRs, some interactions lead to NK inhibition.

NK cells also express the Fc receptor CD16, or FcγRIII. CD16 binding of IgG antibodies bound to target cells activates NK cells, leading to cytokine production and granule release in a process known as antibody-dependent cellular cytotoxicity (ADCC). However CD16 binding to non-antigen bound IgG (monomeric IgG), does not lead to NK activation.

(Introduction to NK92 cells) NK-92 is a NK-like cell line which is distinguished from NK cells by expressing most, but not all, major NK receptors and markers. Importantly, NK-92 cells do not express the Fc receptor for NK cells CD16. NK-92 cells also lack expression of NKp44 and NKp46, activating receptors of the NCR family As a result, although the NK-92 cells are cytotoxic to a significantly broader spectrum of tumor and infected cell types than are NK cells, it cannot potentiate the anti-tumor and anti-infection effects of endogenous or exogenous antibodies due to the absence of CD16 receptor and lack of ADCC induction. However, NK-92 cells have distinct advantages over autologous NK cells used in therapy. NK-92 cells are well defined, grow in continuous culture, and do not have to be purified from mixed lymphocyte populations prior to manipulation or use. Additionally, donor NK or NK-92 cells used in therapy do not need to be HLA matched to recipients as exogenous T cell or T cell lines do, thus enabling a broader use of these cells over T cell based therapies.

(Introduction to NK-CAR, CAR-T NK92-CAR) Chimeric antigen receptors (CAR) are engineered proteins composed of an extracellular receptor region fused to an intracellular signaling region. Normally these regions are from different proteins, however they can also be designed de novo. First used in T cells, CARs can be utilized to activate lymphocytesin which they are expressed. CAR expressing T cells have utilized single-chain variable fragments (scFV) fused to intracellular signaling domains, normally the zeta chain of CD3ζ (CD3ζ). Later developments have included secondary co-stimulatory signals, such as CD28 and CD137, to enhance T cell activation. CAR constructs have also been applied to NK cells, most notably in the use of NK-92 CD19-CAR expression for treatment of CD19+ B cell tumors, which have also been treated with T cell CD19-CARs.

(Introduction to CD19) CD19 (also known as B lymphocyte antigen) is a marker present on B cells from their earliest differentiation throughout their development, and only lost on B cell plasma cells. It is found on few other cell types. B cell derived lymphomas are often CD19+, and CD19 signaling may play a role in driving growth of these cancers. As such CD19 expression can be used as a target for both identification and treatment of these tumors. CD19-CAR-T and CD19-CAR-NK cells have been used to treat CD19+ B cell lymphomas, however these treatments do not cure all patients with CD19+ lymphomas. Current CD19-CAR therapy relies on first generation CARs utilizing a single activating signal, usually from CD3ζ, leaving a need for new designs for CAR expression to treat these patients.

(Introduction to Ganglioside GD2) Ganglioside G2 (GD2) is a sialic acid-containing glycosphingolipid, thought to play an important role in signal transduction as well as cell adhesion and recognition. Its expression is primarily restricted to the central nervous system, peripheral nerves, and skin melanocytes in normal fetal and adult tissues. Expression has also been described in the stromal component of some normal tissues and in the white pulp of the spleen. More importantly, GD2 is expressed at clinically relevant levels in certain cancer types—including lung, neuroblastoma, prostate, melanoma, bone & soft tissue sarcoma.

(Introduction to co-stimulatory signal, including CD28) Costimulatory signals, including CD28, CD137 (4-1BB), and CD134 (OX40), are vital for full and sustained activation of many lymphocytes. On T cells, CD28 co-stimulation during T cell activation leads to increased levels of cytokine production and proliferation. T cell activation lacking co-stimulatory signaling can result in anergy, a T cell state of unresponsiveness to further signaling. Co-stimulation of activated T cells through CD137 and CD134 prolongs and enhances the T cell response, promoting cell survival in addition to cytokine production and cytolitic activity. These receptors (CD28, CD137, and CD134) are also co-stimulatory receptors for NK cell activation. In particular, CD137 upregulation after CD16 engagement on NK cells enhances the ADCC killing of target cells.

(Introduction to CD3Z) The T cell receptor (TCR) is a complex signaling molecule composed of many individual protein chains, including the TCRα/β or γ/6 chains, and CD3's δ, ϵ, ζ, and γ. Of these chains, only the CD3ζ chains contain significant cytoplasmic signaling domains, and CD3ζ mediates signaling from the TCR to downstream activation pathways through its association with the protein kinase ZAP-70 (zeta chain associated protein kinase 70). Thus CD3ζ serves as the crucial intra-cellular signaling domain for signal transduction through the TCR. CD3ζ is a non-membrane spanning protein, and has been associated with CD16, where it mediates CD16 signaling and activation. In addition to its interaction with both the TCR and CD16, CD3ζ mediates the intracellular signaling of many other cell surface receptors.

(Introduction to 2B4) 2B4 (CD244) is a co-stimulatory receptor expressed on both NK cells and CD8+ T cells. It targets a non-MHC like molecule (CD48) expressed on hematopoietic cells, including B and T cells, as well as on activated monocytes and granulocytes. Activation of 2B4 by binding of its ligand on target cells leads to NK (or T cell) activation, and target killing. In NK cells, this activation can be inhibited by other receptor interactions, primarily between inhibitory NK receptors and HLA. 2B4 ligand binding and activation alone is not sufficient for NK activation, however it enhances activation by primary stimulation through other activating receptors. In this context, 2B4 signaling may recruit intracellular signaling molecules that enhance NK cell activation by other surface receptors.

Studies have shown promising effects of engineered T cell or NK92 cell with CD19-CAR expression in treating CD19+ B cell tumors. However, not all the patients are effectively treated. Therefore, there is a need for new designs of CAR constructs for NK cells.

### SUMMARY

The invention provides natural killer cells, including the NK-92 and other NK cell lines, transformed to express a novel chimeric antigen receptor (CAR) on the cell surface wherein the CAR comprises (preferably, from the N terminal to C terminal ends, though the first two domains could be moved) an antigen binding domain, a transmembrane domain, a costimulatory signaling region, and a CD3 zeta signaling domain. Preferably, the cell also expresses an export signal domain, preferably at its N terminus though it could be moved, during biosynthesis of the CAR. Optionally, the cell may also express spacer sequences.

The invention also relates to isolated nucleic acid molecule encoding a Chimeric Antigen Receptor (CAR) construct, wherein the CAR comprises (preferably, N to C terminus) an antigen binding domain, a transmembrane domain, a costimulatory signaling region, a CD3 zeta signaling domain; and to a second isolated nucleic acid molecule expressing a CAR construct comprising (preferably, N to C terminus) an export signal domain, an antigen binding domain, a transmembrane domain, a costimulatory signaling region, and a CD3 zeta signaling domain.

In one embodiment of the invention, the antigen binding domain is a CD19 domain, including a signal peptide sequence of SEQ ID NO: 4 (encoded by the nucleic acid sequence of SEQ ID NO: 3), and an antigen binding domain of SEQ ID NO: 6 (encoded by the nucleic acid sequence of SEQ ID NO: 5). In another preferred embodiment, the antigen binding domain is a Ganglioside G2 (“GD2”) binding domain (not a CD19 domain), in the form of an scFv construct which binds GD2 (SEQ ID NO: 13-16).

The construct preferably includes a linker sequence as in SEQ ID NO: 8, encoded by the nucleic acid sequence of SEQ ID NO: 7. The construct also preferably includes a transmembrane domain and costimulatory signaling region of CD-244 (aka 2B4), as in SEQ ID NO: 10, encoded by the nucleic acid sequence of SEQ ID NO: 9.

The construct also preferably includes a signaling domain which is the CD3 zeta domain, as in SEQ ID NO: 12, encoded by the nucleic acid sequence of SEQ ID NO: 11.

The amino acid sequence of one preferred embodiment of the invention is SEQ ID NO: 2, encoded by the nucleic acid sequence of SEQ ID NO: 1. In another preferred embodiment, the amino acid sequence of SEQ ID NOs: 13 to 16 (GD2-binding scFv with a leader) replace the Signal-CD19-scFv portion of sequence of SEQ ID NO: 2.

For all amino acid and nucleic acid sequences of the preferred constructs and also, for all other constructs and sequences shown, the invention includes amino acid and nucleic acid sequences 70% or more identical thereto, or more preferably, with 90% or more identity, or even more preferably, with 95% or more or 99% or more identity to these amino acid and nucleic acid sequences,

In yet another aspect, the invention is directed to methods of treating a subject having a tumor or other lesion or lymphoma. Said method may include steps of administering at least one type of antibody to the subject, wherein said antibody specifically binds to the tumor or the lesion. The method includes administering the NK cells that are modified to express the CAR to the subject, and which interact with either a cell surface marker on tumorous cells or with said antibodies. A therapeutic response is indicated by a reduction in the tumor or lesion or reductions in tumorous cells.

### BRIEF DESCRIPTION OF DRAWINGS AND SEQUENCES

The file of this patent contains drawings (including the sequence listing) in color. Copies of this patent or patent application publication with the color drawing(s) will be provided by the Office upon request and payment of the necessary fee.

FIG. 1 illustrates the TCR construct and conjugated with scFv.

FIG. 2 illustrates some known NK cell co-stimulatory receptors and their relative positioning in a cell with some known known NK cell co-stimulatory receptors used in CAR, as well as some of the proteins/cytokines they are believed to activate.

FIG. 3 shows (first construct) an NK-CAR construct with a GD2 scFv, and below that, several different NK-CAR constructs with CD19 scFvs, which each include one of several different co-stimulatory regions, which were then inserted into retroviral vectors (using the standard protocol below), and used to transform NK92 cells.

FIG. 4 shows the transfection efficiency of NK92 cells transfected with one of the constructs from FIG. 3 (CD19-IgEγ-2B4-CAR, CD19-2B4-CD3ζ-CAR, CD19-IgEγ-CAR or CD19-CD28-CD3ζ-CAR) and incubated for 72 hrs. The transfection efficiency was analyzed by staining with FITC conjugated rabbit anti-mouse IgG Fc antibody to detect the common IgG4 chains of these constructs (where the Y-axis, SSC, is side scatter).

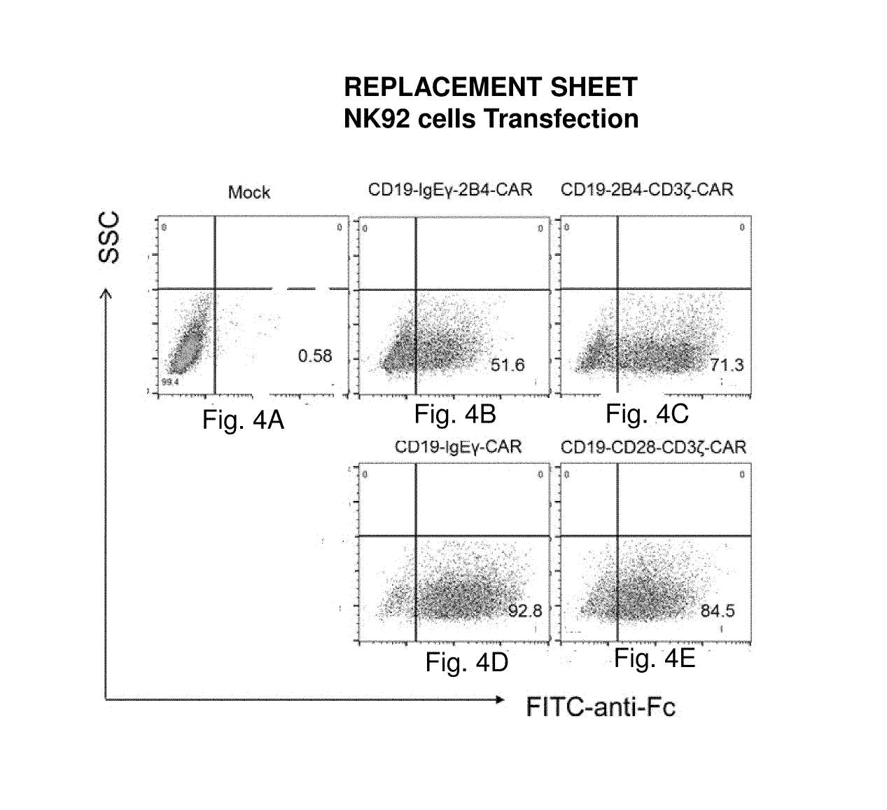

FIG. 5 shows CAR transfected NK92 cells sorted, after staining with FITC conjugated rabbit anti-mouse IgG Fc antibody (as in FIG. 4).

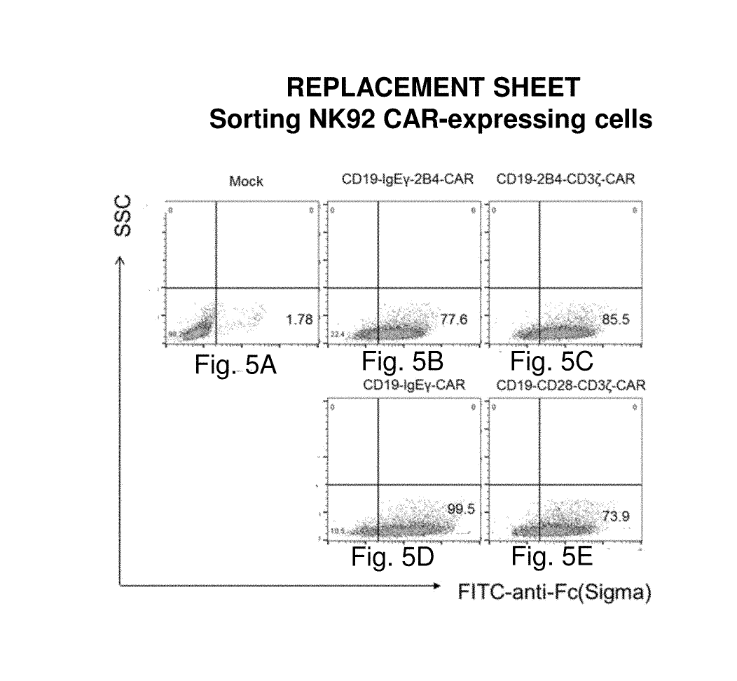

FIG. 6 illustrates that CD19-2B4-CD3ζ-CAR-NK92 is superior to other CD19-CAR-modified NK92 cell lines in inducing Raji tumor lysis. Raji cells were co-cultured with NK92-CAR transfected cells at NK92:Raji ratios of 1:3 or 1:2. Raji cell lysis was analyzed after 24 hours by staining with APC-anti-CD19 (for Raji cells) and FITC-anti-CD56 (NK92 cells). Results show NK92 cells transfected with CD19-2B4-CD3ζ-CAR vector have significantly more tumor lysis activity compared to NK92 cells transfected with other CD19-CAR vectors: CD19-CD28-CD3ζ-CAR, CD19-IgEγ-CAR, and CD19-IgEγ-2B4-CAR. Isolated cell population and numbers indicate viable Raji cells.

FIG. 7 illustrates that CD19-2B4-CD3ζ-CAR-NK92 is superior to other CD19-CAR-modified NK92 cell lines in inducing Daudi tumor lysis. Daudi cells were co-cultured with NK92-CAR transfected cells at NK92:Daudi ratios of 1:4 or 1:8. Daudi cell lysis was analyzed after 24 hr by staining with APC-anti-CD19 (for Daudi cells) and FITC-anti-CD56 (NK92 cells). Results show NK92 cells transfected with CD19-2B4-CD3ζ-CAR vector have significantly more tumor lysis activity comparing NK92 cells transfected with other CD19-CAR vectors (CD19-CD28-CD3ζ-CAR, CD19-IgEγ-CAR, CD19-IgEγ-2B4-CAR). Isolated cell population and numbers indicate viable Daudi cells.

FIG. 8 illustrates that irradiated CD19-2B4-CD3ζ-CAR-NK92 retains superior tumor killing ability compared to other CD19-CAR-modified NK92 cell lines. Raji cells were co-cultured with irradiated or un-irradiated NK92-CAR transfected cells at NK92:Raji cell ratios of 1:4 or 1:8. Raji cell lysis was analyzed by staining with APC-anti-CD19 (for Raji cells) and FITC-anti-CD56 (NK92 cells). Results show that NK92 cells transfected with CD19-2B4-CD3ζ-CAR vector have significantly more tumor lysis activity compared to NK92 cells transfected with other CD19 CAR vectors (CD19-CD28-CD3ζ-CAR, CD19-IgEγ-CAR, CD19-IgEγ-2B4-CAR). Moreover, irradiated CD19-2B4-CD3ζ-CAR-NK92 retains superior tumor killing ability compared to other CD19-CAR-modified NK92 cell lines. Isolated cell population and numbers indicate viable Raji cells.

FIG. 9 shows that a bi-specific antibody binding and linking GD2 and CD3 eradicated tumor cells over 48 hours in pancreatic and neuroblastoma cell lines. In the pancreatic cell line panel, GD2-positive JF-GFP tumor cells were incubated with human PBMC in the presence of control reagent (left) or the bi-specific antibody (right). In the neuroblastoma cell line panel, GD2-positive LAN-1-GFP tumor cells were incubated with human PBMC in the presence of control reagent (left) or bi-specific antibody or control (right).

FIG. 10 indicates the important and functional regions of the CD19-scFv antibody domain, where: the immunoreceptor tyrosine-based switch motifs (ITSM), which have sequences: T-x-Y-x-x-[V/I], are underlined, because the phosphorylated tyrosines, designed “Y*,” have demonstrated importance for activation of 2B4. Sequence mutations in these regions, particularly resulting in changes to the expression of threonine (T), tyrosine (Y), or valine (V)/isoleucine (I) are likely to prevent activation of 2B4, and to prevent activation of CARs incorporating the 2B4 transmembrane and cytoplasmic domains. The presumed cytosolic region is also indicated.

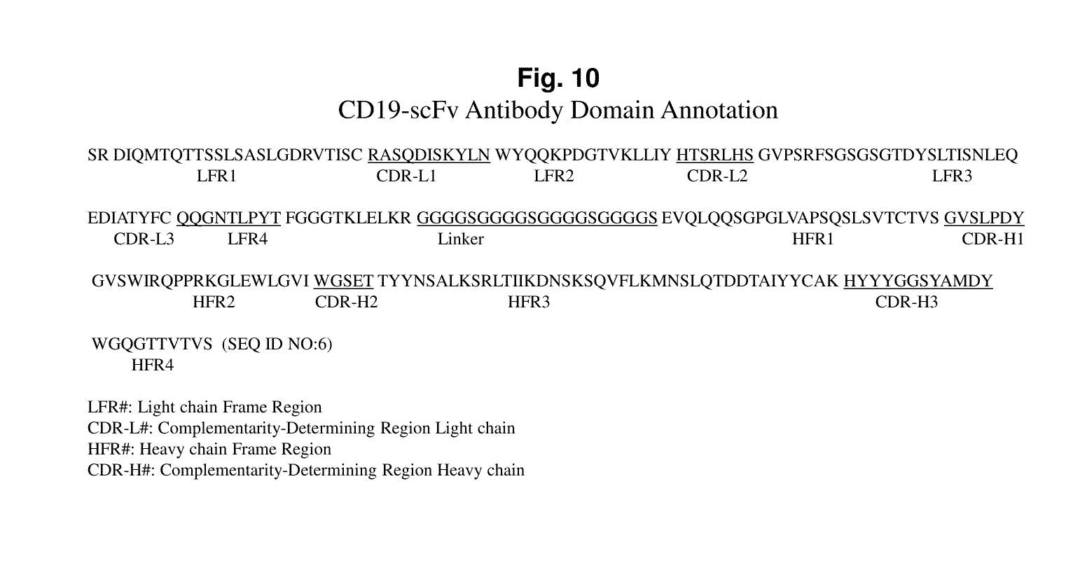

FIG. 11 indicates the important and functional regions of the 2B4 domain, where: the immunoreceptor tyrosine-based switch motifs (ITSM), which have sequences: T-x-Y-x-x-[V/I], are underlined, because the phosphorylated tyrosines, designed “Y*,” have demonstrated importance for activation of 2B4. Sequence mutations in these regions, particularly resulting in changes to the expression of threonine (T), tyrosine (Y), or valine (V)/isoleucine (I) are likely to prevent activation of 2B4, and to prevent activation of CARs incorporating the 2B4 transmembrane and cytoplasmic domains. The presumed cytosolic region is also indicated.

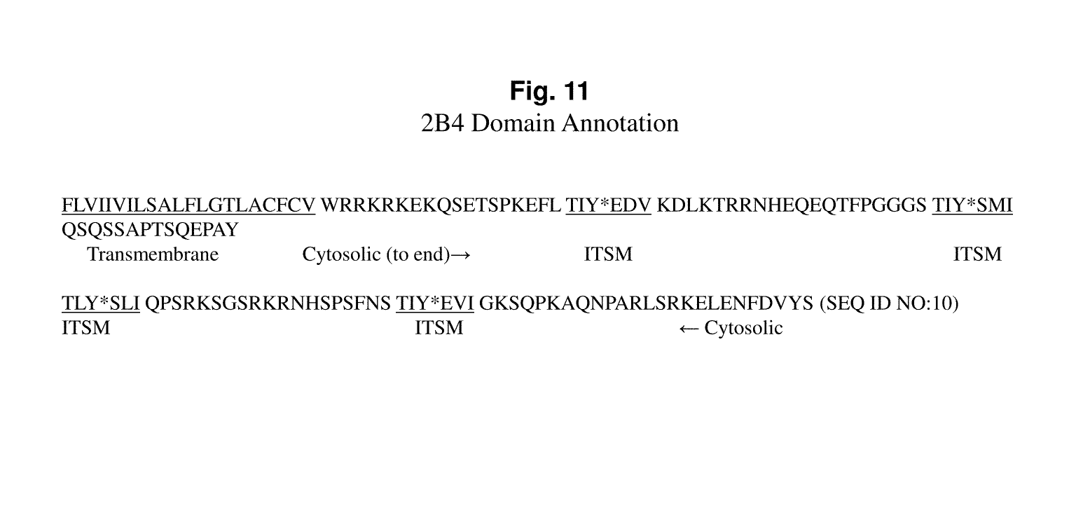

FIG. 12 indicates the important and functional regions of the CD3ζ domain. The immunoreceptor tyrosine-based switch motifs (ITSM), which have sequences: T-x-Y-x-x-[V/I], are underlined, because the phosphorylated tyrosines, designed “Y*,” have demonstrated importance for activation of CD3ζ.

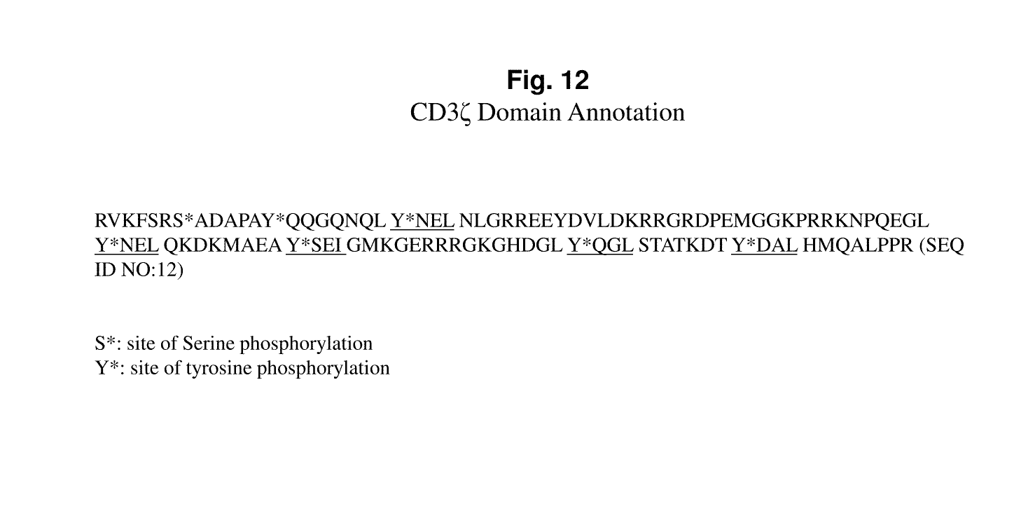

Sequence mutations in these regions, particularly resulting in changes to the expression of threonine (T), tyrosine (Y), or valine (V)/isoleucine (I) are likely to prevent activation of CD3ζ, and to prevent activation of CARs incorporating CD3ζ.

### SEQUENCE LISTING GUIDE

SEQ ID NO:1 is the DNA sequence of the Signal-CD19-scFv-IgG4-CH2CH3-2B4-CD3ζ construct.

SEQ ID NO:2 is the amino acid sequence of the Signal-CD19-scFv-IgG4-CH2CH3-2B4-CD3ζ construct.

SEQ ID NO:3 is the DNA sequence of the antibody heavy chain signal peptide portion of SEQ ID NO:1.

SEQ ID NO:4 is the amino acid sequence of the antibody heavy chain signal peptide portion of SEQ ID NO:2.

SEQ ID NO:5 is the DNA sequence of the CD19-scFv portion of SEQ ID NO:1.

SEQ ID NO:6 is the amino acid sequence of the CD19-scFv portion of SEQ ID NO:2.

SEQ ID NO:7 is the DNA sequence of the IgG4-CH2CH3 portion of SEQ ID NO:1.

SEQ ID NO:8 is the amino acid sequence of the IgG4-CH2CH3 portion of SEQ ID NO:2.

SEQ ID NO:9 is the DNA sequence of the 2B4 transmembrane domain and cytoplasmic domain portion of SEQ ID NO:1.

SEQ ID NO:10 is the amino acid sequence of the 2B4 transmembrane domain and cytoplasmic domain portion of SEQ ID NO:2.

SEQ ID NO:11 is the DNA sequence of the CD3ζ cytoplasmic domain portion of SEQ ID NO:1.

SEQ ID NO:12 is the amino acid sequence of the CD3ζ cytoplasmic domain portion of SEQ ID NO:2.

SEQ ID NO:13 is the amino acid sequence of the leader portion of the construct: Leader-VH14g2a-(SG4SG2)-VL14g2a, which can replace the Signal-CD19-scFv portion of the construct in SEQ ID NO:1, in one embodiment.

SEQ ID NO: 14 is the amino acid sequence of the VH14g2a portion of the construct: Leader-VH14g2a-(SG4SG2)-VL14g2a, which can replace the Signal-CD19-scFv portion of the construct in SEQ ID NO:1, in one embodiment.

SEQ ID NO: 15 is the amino acid sequence of the SG4SG2 portion of the construct: Leader-VH14g2a-(SG4SG2)-VL14g2a, which can replace the Signal-CD19-scFv portion of the construct in SEQ ID NO:1, in one embodiment.

SEQ ID NO: 16 is the amino acid sequence of the VL14g2a portion of the construct: Leader-VH14g2a-(SG4SG2)-VL14g2a, which can replace the Signal-CD19-scFv portion of the construct in SEQ ID NO:1, in one embodiment.

SEQ ID NO: 17 is the sequence of a forward primer for amplifying an insert.

SEQ ID NO: 18 is the sequence of a reverse primer for amplifying an insert.

### DETAILED DESCRIPTION

While the present invention is susceptible of embodiment in many different forms, there are shown in the drawings, and described herein in detailed specific embodiments and examples thereof, with the understanding that the disclosure is to be considered as an exemplification of the principles of the invention and is not intended to limit the invention to the specific embodiments illustrated.

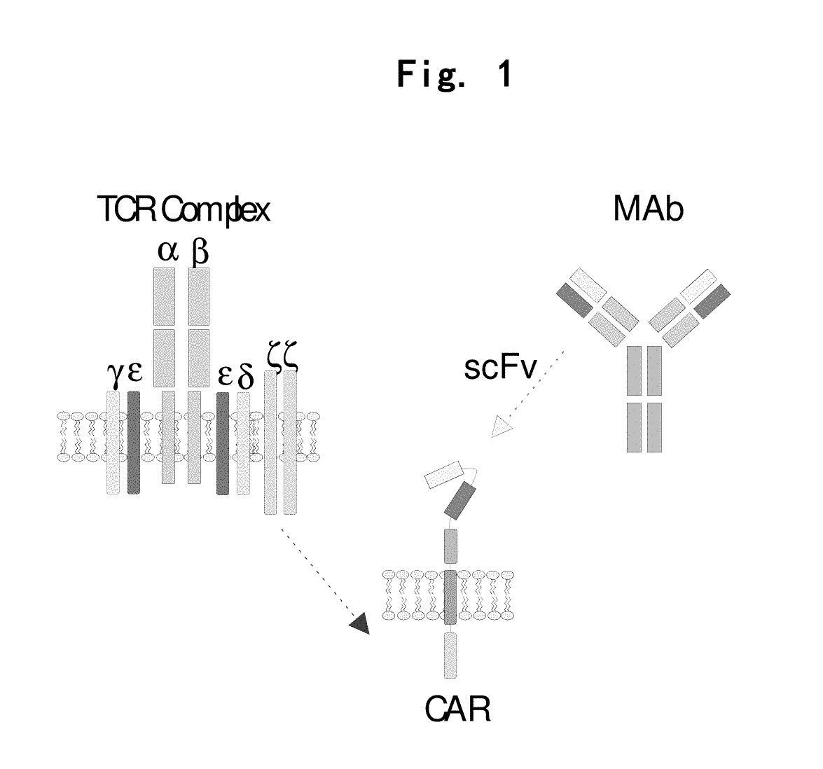

The present invention provides a modified NK cell including NK92 that expresses a novel chimeric antigen receptor (CAR) on the surface of the cell. FIG. 1 shows the structure of an scFv domain (taken from a monoclonal antibody, MAb), the position in a cell membrane of a T cell receptor complex (TCR), and in the lowermost panel, an scFv-chimeric antigen receptor (CAR) construct of the invention positioned in a cell membrane.

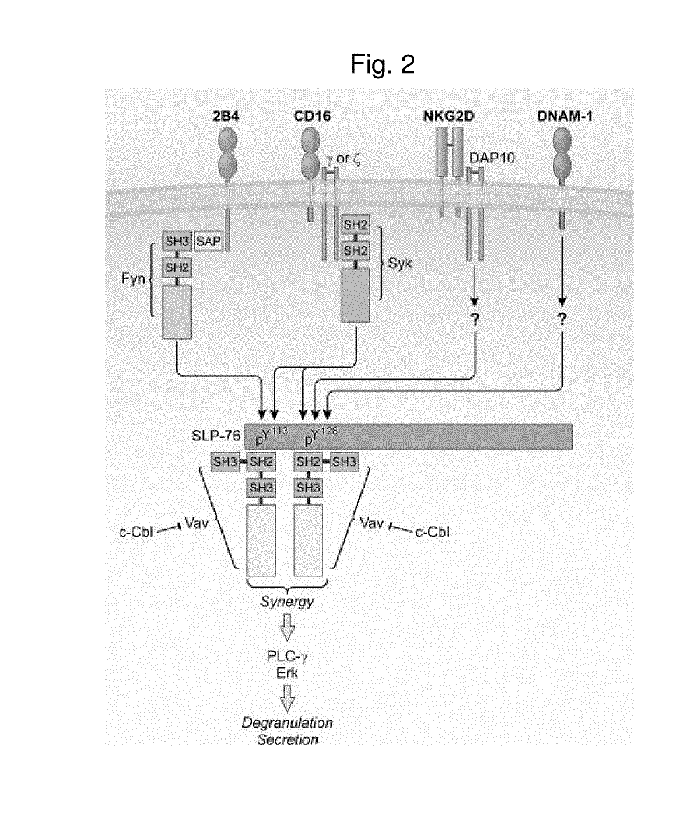

FIG. 2 depicts some known NK cell co-stimulatory receptors and their relative positioning in a cell with some known NK cell co-stimulatory receptors used in CARs, as well as some of the proteins/cytokines they are believed to activate. IgE-γ and CD3ζ are the major signaling entities for activation of NK cells. However to achieve full activation and enhanced antitumor effects, NK cells are preferably co-stimulated with secondary entities, like: 2B4, NKG2D/DAP10, and DNAM-1. The co-stimulation and activation can be cause of the observed enhanced NK cell killing function.

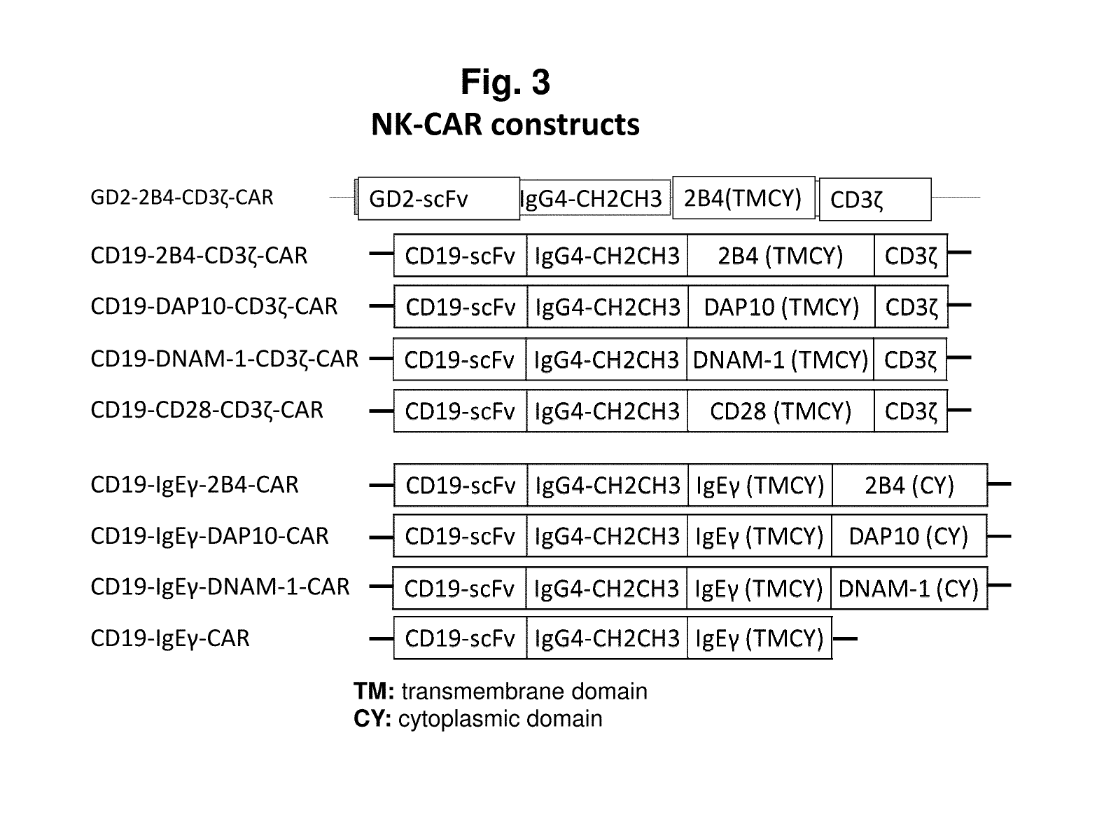

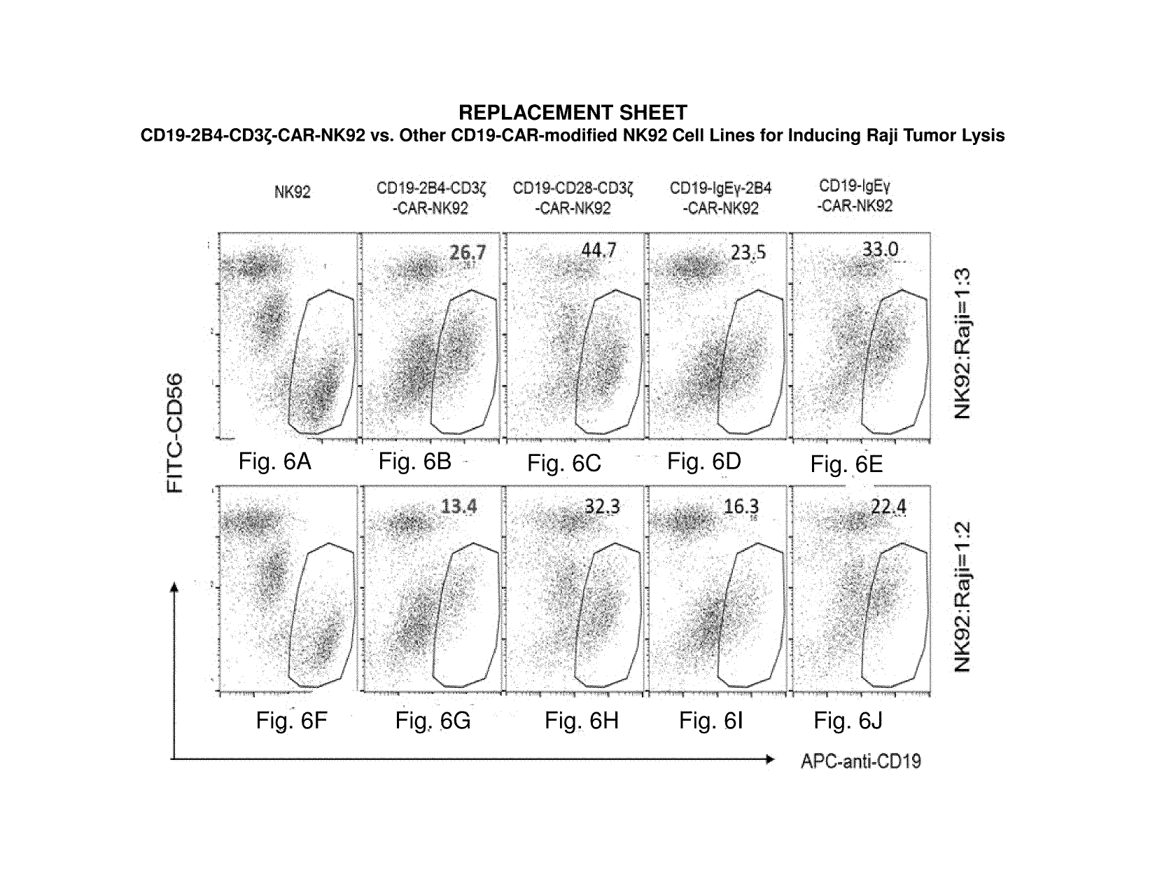

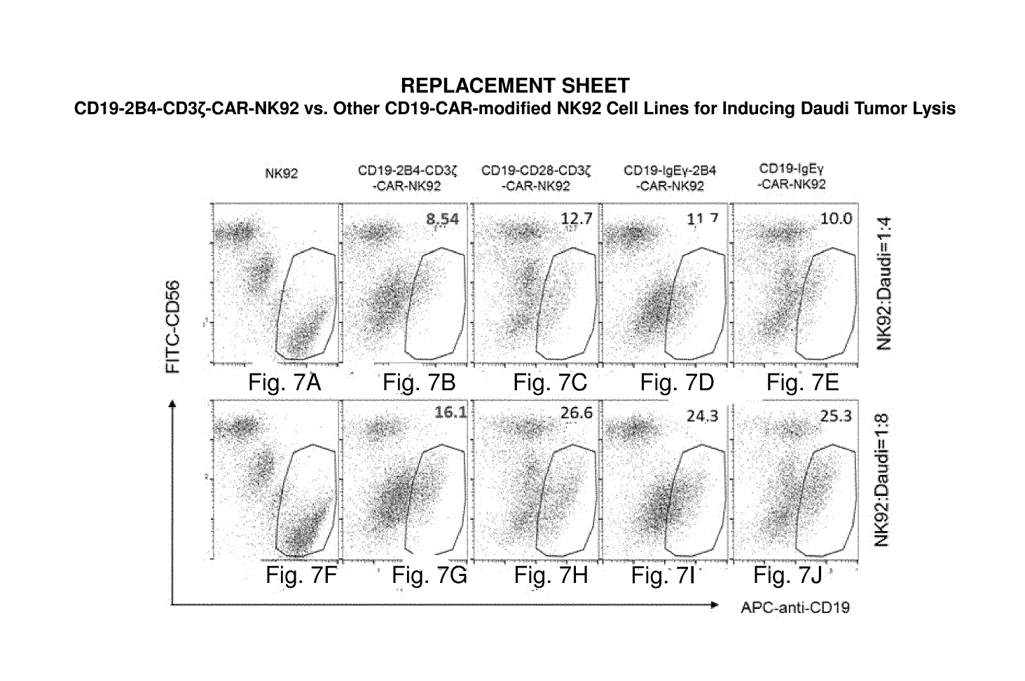

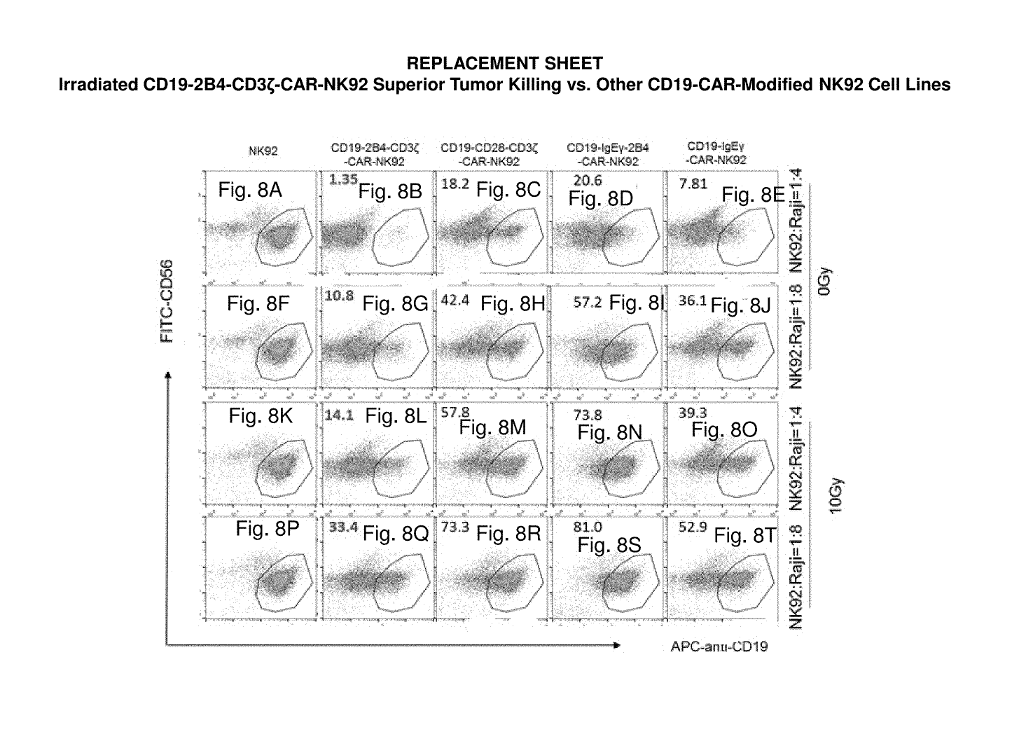

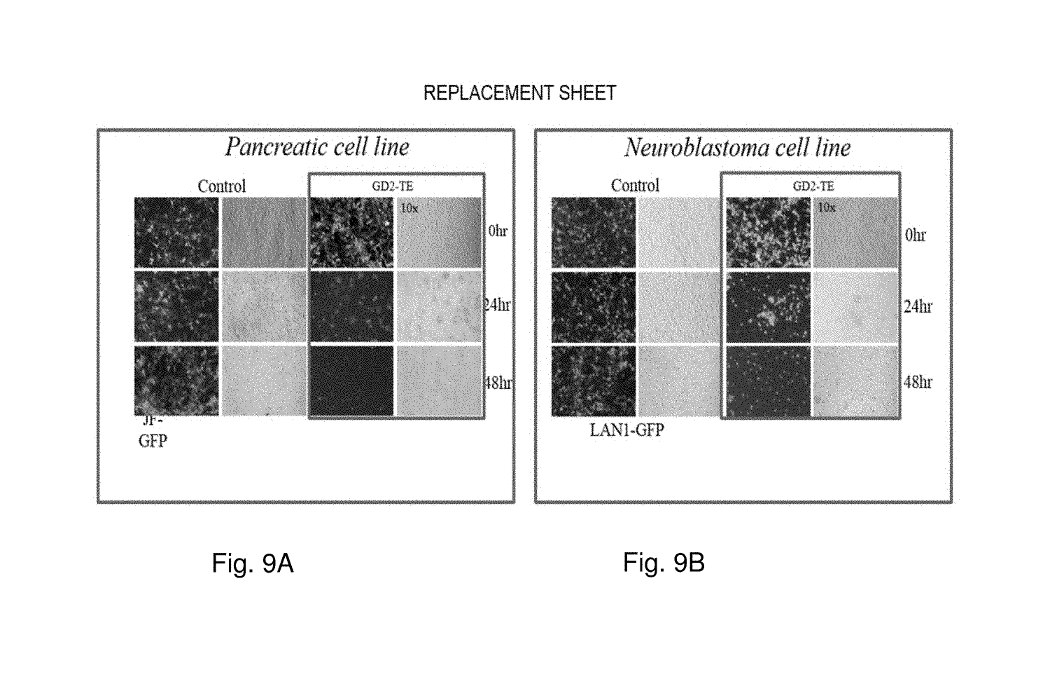

FIG. 3 depicts the portions and relative positions (N terminus to C terminus) of a number of the CAR constructs of the invention which are expressed in NK cells. The uppermost construct (GD2-2B4-CD3ζ-CAR) was not transfected into NK cells. But such construct is believed to also be highly effective in cell killing, based on results shown in FIG. 9—where a bispecific antibody targeting GD2 and CD3 (on CD3-bearing T cells) was shown to eradicate tumors within 48 hours in two different cell types—in combination with the other similarities between the uppermost construct and the next-highest construct (CD19-2B4-CD3ζ-CAR) in FIG. 3, which was the most effective construct tested for tumor cell killing (see FIGS. 6-8).

The invention also includes methods of therapeutic treatment of genetically modified NK cells expressing a CAR that comprises an antigen binding domain that binds to CD19 as described herein; and wherein, the CAR of such genetically modified NK92 includes the following regions (N to C terminus): an antigen binding domain, a transmembrane domain, a costimulatory signaling region, and a CD3 zeta signaling domain. More preferably, during biosynthesis, the CAR includes (N to C terminus): a region representing all or a portion or variant of an export signal peptide, a region representing all or a portion or variant of CD19-scFv, a region representing all or a portion or variant of IgG4-CH2CH3, a region representing all or a portion or variant of 2B4, and a region representing all or a portion or variant of CD3ζ; wherein the term” all or a portion or variant of” herein means at least 70%, or at least 75%, or at least 80%, or at least 85%, or at least 90%, or at least 95%, or at least 99%, or 100% sequence identify with the sequences, or a portion of them, for each of the export signal peptide and the other foregoing regions (or other regions below); where the entire construct amino acid sequence is in SEQ ID NO: 2; and amino acid SEQ ID NOS.: 4, 6, 8, 10, 12 respectively show the sequence of the various portions of the construct Signal-CD19-scFv-IgG4-CH2CH3-2B4-CD3ζ (SEQ ID NO: 2), moving left to right. The nucleotide sequences encoding each of the foregoing regions (SEQ ID NOS.: 3, 5, 7, 9, 11, respectively) and all or a portion or variant of those sequences, are also within the scope of the invention.

As noted, the uppermost construct (GD2-2B4-CD3ζ-CAR) in FIG. 3 is expected to be highly effective in tumor cell killing. The amino acid sequence of a preferred such construct is from combining the sequences shown in SEQ ID NOS.: 13, 14, 15 and 16, and can also include all or a portion or variant of the entire uppermost construct in FIG. 3, or of a sequence in any of SEQ ID NOS.: 13, 14, 15 and 16, or any of the corresponding nucleic acid sequences to any of the amino acid sequences in SEQ ID NOS.: 13, 14, 15 and 16.

Accordingly, the CAR is encoded by a nucleic acid sequence of SEQ ID NO: 1, or by the nucleic acid sequence corresponding to the combination of the amino acid sequences in SEQ ID NOS.: 13, 14, 15 and 16 (“preferred encoding nucleic acids”). Alternatively, the CAR is encoded by: a nucleic acid sequence that have at least one, but no more than thirty modifications of the nucleic acid sequence of either of the preferred encoding nucleic acids; or, a nucleic acid sequence having 95% to 99% identity to either of the preferred encoding nucleic acids. Extracellular linking domains may also be encoded by the preferred encoding nucleic acids.

### EXAMPLE I

### Retroviral Vector Construction & NK92 Cell Transfection

The general protocol for construction of the CD19-2B4-CD3ζ CAR and transfection of NK cells, is as below. The construct in the next to uppermost panel in FIG. 3 (GD2-2B4-CD3ζ-CAR) is made using a similar protocol. Different CAR vector constructs may follow different construction protocols depending on the ability to synthesize the CAR insert, or the ability to construct the insert from existing fragments of template DNA by restriction enzyme digestion and ligation; as well as different transfection protocols. All such changes to the commercial products used, variations of the protocol, and changes to the protocol are standard practice in molecular biology.

Construction of plasmid containing CAR vector:
1) Genomic insert SEQ ID NO: 1 (CD19-IgG4CH2CH3-2B4-CD3ζ) was synthesized by Genescript.
2) The insert was amplified utilizing the following forward and reverse primers, incorporating the restriction site Ncol at the 5′, and SphI at the 3′ end of the gene insert.

FRWRD:
(SEQ ID NO: 17)
ACCATGGAGTTTGGGCTGAGC;
RVRS:
(SEQ ID NO: 18)
GCATGCTAACGCGTTTAGCGAGG.

3) Plasmid pSFG-GFP, and PCR amplified gene insert were digested with Ncol and SphI.
4) Digestion products were purified by gel electrophoresis
5) Purified plasmid and gene insert were ligated by compatible ends.
6) Ligation product was transfected into E. coli DH5α
7) DH5α were grown on ampicillin containing agar to select transfected colonies.
8) Colonies were screened for correct sequence and orientation of the insert.
9) Plasmid (pSFG-CD19-IgG4CH2CH3-2B4-CD3ζ) was purified from the screened DH5a colony.

Production of retrovirus containing CAR vector:
1) HEK293 cells were transfected with pSFG plasmid containing the CAR vector, as well as retroviral production plasmids pRD114 and pEQ-PAM3(-E), using OptiMEM (Thermofisher) and GeneJuice (EMDMillipore).
2) Transfected HEK293 cells were cultured for 48 hours.
3) Supernatant from transfected cells was harvested by centrifuging to remove debris, filtered through a 0.45 μm filter.
4) Purified retrovirus was frozen for future use.

Retroviral transduction of NK92 cells with CAR vector:
1) Coat a non-tissue culture treated 24-well plate with retronectin (7 μl/well in 1 ml of Phosphate Buffered Saline-PBS), incubate O/N at 4° C.
2) Remove plate from 4° C., remove retronectin solution, and wash wells once with complete DMEM (DMEM supplemented with 10% FBS, Pen/Strep (100U/mL), and 1mM Sodium Pyruvate).
3) Add 500-700 μL of viral supernatant to well.
4) Place plate into incubator for 30 min
5) Remove viral supernatant; repeat steps 3-4 1 ×.
6) Remove viral supernatant and replace with 1.5 mL of new viral supernatant.
7) Add 500 μL (2.5×10̂5 cells) of NK92 cells in complete DMEM.
8) Spin the plate at 1000 g for 30 minutes then put the plate back in the incubator.
9) Feed cells with new media every 2-3 days. Examine cells for transduction (by expression of CD19-CAR) and sub-culture transduced cells for continuous growth. Freeze transduced cells.

The modified NK cells of the invention are administered a patient in an amount effective for treatment of a cancer. Preferably, the transformed NK cells are administered by IV infusion. However, a variety of dosing regimes, administration routes including any parenteral administration, e.g., subcutaneous (s.c), intravenous (i.v.), intramuscular (i.m.), or intrasternal injection, intratumoral, or infusion techniques, can be used to deliver transformed NK cells.

Preferably, the patient receives 1×10̂5-5×10̂9 transformed NK cells/meter squared of body surface area. More preferably, the patient receives 1-5×10̂9 cells/meter squared of body surface area. More preferably, the patient receives the cells over a dosing period of 15 minutes to 2 hours.

The patient can receive up to three doses of transformed NK cells per week for at least one month, and more preferably, receives at least 3 doses per week for at least 3-6 months. The patient can receive combination therapies with other products as described for example in International Publication No. WO2015142675 (incorporated by reference). The patient can also receive any formulation described in WO2015142675. The wide variety of dosages, administration routes, dosing regimes, combination therapies and other variants of the specific procedures described herein are set forth in WO2015142675 and incorporated by reference herein.

Also disclosed are methods of cryopreservation and cryopreserved transformed NK cells. The cryopreservation medium used in the method can include a cryoprotectant (preferably either DMSO or polyvinylpyrrolidone (PVP)), and insulin-like growth factor 1.

The specific methods and compositions described herein are representative of preferred and other embodiments are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. Thus, for example, in each instance herein, in embodiments or examples of the present invention, any of the terms “comprising”, “including”, containing”, etc. are to be read expansively and without limitation. The methods and processes illustratively described herein suitably may be practiced in differing orders of steps, and that they are not necessarily restricted to the orders of steps indicated herein or in the claims. It is also noted that as used herein and in the appended claims, the singular forms “a,” “an,” and “the” include plural reference, and the plural include singular forms, unless the context clearly dictates otherwise. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A nucleic acid construct encoding a Chimeric Antigen Receptor (CAR) for transfecting into and modifying an NK cell wherein, the CAR comprises the following regions: a region representing all or a portion or variant of an export signal peptide; a region representing all or a portion or variant of CD19-scFv; a region representing all or a portion or variant of IgG4-CH2CH3 or one of IgG4, IgG2, IgG1, IgG3, IgM, IgE, CD8 linked with the constant heavy chains CH2 and/or CH3 of one of IgG4, IgG2, IgG1, IgG3, IgM, IgE; a region representing all or a portion or variant of 2B4; and a region representing all or a portion or variant of CD3ζ.

2. The nucleic acid construct of claim 1 comprising SEQ ID NO:1 or a sequence at least 70% identical thereto.

3. A transformed NK cell which expresses the nucleic acid construct of claim 1.

4. The transformed NK cell of claim 3 wherein the sequence of the expressed CAR is at least 70% identical to SEQ ID NO:2.

5. A nucleic acid construct encoding a Chimeric Antigen Receptor (CAR) for transfecting into and modifying an NK cell wherein, the CAR comprises a combination (from the N to C terminus) of a GD2 and leader region including SEQ ID NO:13, SEQ ID NO:14, SEQ ID NO:15, and SEQ ID NO:16 or a sequence 70% identical to said GD2 and leader region; and a second region representing all or a portion or variant of IgG4-CH2CH3 or one of IgG4, IgG2, IgG1, IgG3, IgM, IgE, CD8 linked with the constant heavy chains CH2 and/or CH3 of one of IgG4, IgG2, IgG1, IgG3, IgM, IgE; a 2B4 region representing all or a portion or variant of 2B4;
and a CD3ζ region representing all or a portion or variant of CD3ζ.

6. A transformed NK cell which expresses the nucleic acid construct of claim 5.

7. The nucleic acid construct of claim 5 wherein said second region is at least 70% identical to SEQ ID NO:8, said 2B4 region is at least 70% identical to SEQ ID NO:10 and said CD3ζ region is at least 70% identical to SEQ ID NO:12.

8. A transformed NK cell which expresses a Chimeric Antigen Receptor (CAR) at least 70% identical to (from the N to C terminus) SEQ ID NO:13, SEQ ID NO:14, SEQ ID NO:15, SEQ ID NO:16, SEQ ID NO:8, SEQ ID NO:10 and SEQ ID NO:12.

9. The nucleic acid construct of claim 2 which is at least 75%, or at least 80%, or at least 85%, or at least 90%, or at least 95%, or at least 99%, identical to the sequence of SEQ ID NO:1.

10. The transformed NK cell of claim 4 wherein the sequence of the expressed CAR is at least 75%, or at least 80%, or at least 85%, or at least 90%, or at least 95%, or at least 99%, identical to the sequence of SEQ ID NO:2.

11. The nucleic acid construct of claim 7 wherein said GD2 and leader region is at least 75%, or at least 80%, or at least 85%, or at least 90%, or at least 95%, or at least 99% identical to the combination of SEQ ID NO:13, SEQ ID NO:14, SEQ ID NO:15 and SEQ ID NO:16; and the second region is at least 75%, or at least 80%, or at least 85%, or at least 90%, or at least 95%, or at least 99% identical to SEQ ID NO:8, said 2B4 region is at least 75%, or at least 80%, or at least 85%, or at least 90%, or at least 95%, or at least 99% identical to SEQ ID NO:10 and said CD3ζ region is at least 75%, or at least 80%, or at least 85%, or at least 90%, or at least 95%, or at least 99% identical to SEQ ID NO:12.

12. The transformed NK cell of claim 3 which is a NK 92 cell.

13. Administering transformed NK cells of claim 12 to a patient in an amount effective for treatment of a cancer or a tumor.

14. The administration of claim 12 wherein patient receives up to 3 doses per week for at least one month.

15. The administration of claim 12 wherein patient receives at least 3 doses per week for at least 3-6 months.

16. The transformed NK cell of claim 4 which is a NK 92 cell.

17. The transformed NK cell of claim 6 which is a NK 92 cell.

18. The transformed NK cell of claim 8 which is a NK 92 cell.

19. The transformed NK cell of claim 10 which is a NK 92 cell.
